(19) United States
(12) Patent Application Publication
(10) Pub. No.: US 2010/0063394 A1
(43) Pub. Date: Mar. 11, 2010

(54) OSTEOPOROSIS PROGRESS STAGE MEASURING DEVICE AND OSTEOPOROSIS PROGRESS STAGE MEASURING METHOD

(75) Inventor: Hiroto Tateno, Kagoshima (JP)

Correspondence Address: ARENT FOX LLP, 1050 CONNECTICUT AVENUE, N.W., SUITE 400, WASHINGTON, DC, 20036, US

(73) Assignee: KAGOSHIMA UNIVERSITY, Kagoshima-shi, Kagoshima (JP)

(21) Appl. No.: 12/514,923

(22) PCT Filed: Nov. 13, 2007
(86) PCT No.: PCT/JP2007/071956

§ 371 Date: May 14, 2009

(30) Foreign Application Priority Data
Nov. 14, 2006 (JP) 2006-308343

(51) Int. Cl. A61B 8/00

(52) U.S. Cl. 600/449

(57) ABSTRACT

An osteoporosis progress stage measuring device is configured such that an ultrasonic wave oscillating section (110) oscillates an ultrasonic wave that gives torsional vibration to a bone part of a subject (200), an ultrasonic wave detecting section (120) detects, through the subject (200), an ultrasonic wave attributed to the torsional vibration propagated through the bone part of the subject (200), a CPU (150) calculates, based on the detected ultrasonic wave attributed to the torsional vibration, an attenuation coefficient (attenuation ratio) and a sound speed of the ultrasonic wave, and calculates, based on the calculated attenuation coefficient and the sound speed, a characteristic indicating the progress stage of osteoporosis (bone age) in the bone part of the subject (200). Accordingly, the progress stage of osteoporosis can be measured quantitatively and adequately without placing a significant burden on a body of the subject.

### TECHNICAL FIELD

The present invention relates to an osteoporosis progress stage measuring device and an osteoporosis progress stage measuring method measuring a progress stage of osteoporosis caused by a decrease in bone mass.

### BACKGROUND ART

Contemporary society is, for better or for worse, a society of vehicle typified by a car or the like, and a decrease in an amount of exercise which is necessary for maintaining health of human being is concerned. It is known that the decrease in the amount of exercise which is necessary for maintaining health of human being causes a significant decrease in bone mass, namely, an exhibition of osteoporosis-like changes.

Further, also by a research of the present inventors, an experimental result is obtained in which osteoporosis-like changes in accordance with a reduction in mechanical load with respect to a femur causes an irreversible organic disorder even after the load is applied again. For example, the present inventors report the following experimental result in non-patent document 1 described below.

Wistar-type male rats each being 7 weeks old and having weight of about 200 g are divided into two groups, which are, a hind-limbs suspended group in which hind limbs of the rats are suspended for 9 weeks and the rats are returned to a cage and raised for 8 weeks, and a control group in which the rats are raised as they are in a cage. After the rats are raised, a femur of hind limb is removed from each of the Wistar-type male rats of the both groups. A result is obtained that, compared to the femur of hind limb of the Wistar-type male rat of the latter control group, the femur of hind limb of the Wistar-type male rat of the former hind-limbs suspended group does not exhibit recovery during a sufficient convalescence period under gravity, and it locally has a vulnerability.

Specifically, the aforementioned experimental result of non-patent document 1 indicates that when the reduction in mechanical load with respect to a bone is continuously conducted, osteoporosis-like changes in accordance with the reduction in mechanical load do not recover to the original state even after the load is applied again. Accordingly, it is very important to enable to easily grasp a progress stage of osteoporosis at each time, as a measure for suppressing an increase in medical costs and care costs in today's aging society.

non-patent document 1: Proceedings of symposium on ultrasonic electronics, vol. 26 (issued on Nov. 16, 2005), P. 157

### SUMMARY OF THE INVENTION

Conventionally, as a method of measuring the progress stage of osteoporosis, a method using X-rays, a method using ultrasonic waves, and the like have been devised. Among the above, the method using X-rays has a problem that it places a significant burden on a body of a subject since a measurement is conducted by irradiating X-rays on the subject. The problem becomes more significant particularly when the subject is of advanced age or is a patient who is hospitalized for long periods of time.

On the other hand, the method using ultrasonic waves does not create a problem, as in the method using X-rays, that it places a significant burden on a body of a subject, but, with the use of a conventional method using ultrasonic waves, it has been difficult to quantitatively and adequately measure the progress stage of osteoporosis.

The present invention has been made in view of the above-described problems, and an object thereof is to provide an osteoporosis progress stage measuring device and an osteoporosis progress stage measuring method with which a measurement of progress stage of osteoporosis can be realized quantitatively and adequately without placing a significant burden on a body of a subject.

An osteoporosis progress stage measuring device of the present invention includes: an oscillating unit oscillating an ultrasonic wave that gives torsional vibration to a bone part of a subject and an ultrasonic wave that gives vertical vibration to the bone part; a detecting unit detecting, through the subject, an ultrasonic wave attributed to the torsional vibration propagated through the bone part and an ultrasonic wave attributed to the vertical vibration propagated through the bone part; a sound speed calculating unit calculating a sound speed of the ultrasonic wave attributed to the torsional vibration based on the ultrasonic wave attributed to the torsional vibration detected by the detecting unit, and a sound speed of the ultrasonic wave attributed to the vertical vibration based on the ultrasonic wave attributed to the vertical vibration detected by the detecting unit; and an osteoporosis progress stage characteristic calculating unit calculating a characteristic indicating the progress stage of osteoporosis in the bone part by dividing the sound speed of the ultrasonic wave attributed to the torsional vibration by the sound speed of the ultrasonic wave attributed to the vertical vibration calculated by the sound speed calculating unit.

An osteoporosis progress stage measuring device according to another aspect of the present invention includes: an oscillating unit oscillating an ultrasonic wave that gives torsional vibration to a bone part of a subject; a detecting unit detecting, through the subject, an ultrasonic wave attributed to the torsional vibration propagated through the bone part; an attenuation coefficient calculating unit calculating an attenuation coefficient of the ultrasonic wave attributed to the torsional vibration detected by the detecting unit with respect to the ultrasonic wave oscillated by the oscillating unit; and an osteoporosis progress stage characteristic calculating unit calculating, based on the attenuation coefficient calculated by the attenuation coefficient calculating unit, a characteristic indicating the progress stage of osteoporosis in the bone part.

Further, an osteoporosis progress stage measuring device according to still another aspect of the present invention includes: an oscillating unit oscillating an ultrasonic wave that gives torsional vibration to a bone part of a subject and an ultrasonic wave that gives vertical vibration to the bone part; a detecting unit detecting, through the subject, an ultrasonic wave attributed to the torsional vibration propagated through the bone part and an ultrasonic wave attributed to the vertical vibration propagated through the bone part; a sound speed calculating unit calculating a sound speed of the ultrasonic wave attributed to the torsional vibration based on the ultrasonic wave attributed to the torsional vibration detected by the detecting unit, and a sound speed of the ultrasonic wave attributed to the vertical vibration based on the ultrasonic wave attributed to the vertical vibration detected by the detecting unit; and an osteoporosis progress stage characteristic calculating unit calculating a characteristic indicating the progress stage of osteoporosis in the bone part by determining a Poisson's ratio using the sound speed of the ultrasonic wave attributed to the torsional vibration and the sound speed of the ultrasonic wave attributed to the vertical vibration calculated by the sound speed calculating unit.

An osteoporosis progress stage measuring method according to the present invention includes: an oscillating step of oscillating an ultrasonic wave that gives torsional vibration to a bone part of a subject and an ultrasonic wave that gives vertical vibration to the bone part; a detecting step of detecting, through the subject, an ultrasonic wave attributed to the torsional vibration propagated through the bone part and an ultrasonic wave attributed to the vertical vibration propagated through the bone part; a sound speed calculating step of calculating a sound speed of the ultrasonic wave attributed to the torsional vibration based on the ultrasonic wave attributed to the torsional vibration detected by the detecting step, and a sound speed of the ultrasonic wave attributed to the vertical vibration based on the ultrasonic wave attributed to the vertical vibration detected by the detecting step; and an osteoporosis progress stage characteristic calculating step of calculating a characteristic indicating the progress stage of osteoporosis in the bone part by dividing the sound speed of the ultrasonic wave attributed to the torsional vibration by the sound speed of the ultrasonic wave attributed to the vertical vibration calculated by the sound speed calculating step.

An osteoporosis progress stage measuring method according to another aspect of the present invention includes: an oscillating step of oscillating an ultrasonic wave that gives torsional vibration to a bone part of a subject; a detecting step of detecting, through the subject, an ultrasonic wave attributed to the torsional vibration propagated through the bone part; an attenuation coefficient calculating step of calculating an attenuation coefficient of the ultrasonic wave attributed to the torsional vibration detected by the detecting step with respect to the ultrasonic wave oscillated by the oscillating step; and an osteoporosis progress stage characteristic calculating step of calculating, based on the attenuation coefficient calculated by the attenuation coefficient calculating step, a characteristic indicating the progress stage of osteoporosis in the bone part.

According to the present invention, it is possible to measure a progress stage of osteoporosis quantitatively and adequately without placing a significant burden on a body of a subject.

### BRIEF DESCRIPTION OF THE DRAWINGS

FIG. 1A is a characteristic diagram when an ultrasonic wave is applied to the femur of hind limb of the rat in the control group described in Background Art;

FIG. 1B is a characteristic diagram when an ultrasonic wave is applied to the femur of hind limb of the rat in the control group described in Background Art;

FIG. 2A is a characteristic diagram when an ultrasonic wave is applied to the femur of hind limb of the rat in the hind-limbs suspended group described in Background Art;

FIG. 2B is a characteristic diagram when an ultrasonic wave is applied to the femur of hind limb of the rat in the hind-limbs suspended group described in Background Art;

FIG. 3 is a block diagram showing a schematic configuration of an osteoporosis progress stage measuring device according to an embodiment of the present invention;

FIG. 4A is a schematic diagram showing an example of reference data of osteoporosis progress stage;

FIG. 4B is a schematic diagram showing an example of reference data of osteoporosis progress stage;

FIG. 5 is a diagram for explaining a damped wave of an ultrasonic wave that propagates through a bone part (calcaneus to tibia) of a subject;

FIG. 6 is a schematic diagram showing a schematic configuration of a tibia shown in FIG. 3;

FIG. 7 is a flow chart showing a processing procedure of an osteoporosis progress stage measuring method using the osteoporosis progress stage measuring device according to the embodiment of the present invention;

FIG. 8 is a characteristic diagram showing a phase value and a phase-correlation amplitude value of an ultrasonic wave attributed to torsional vibration propagated through a tibia of a male S who is 22 years old; and

FIG. 9 is a characteristic diagram showing a phase value and a phase-correlation amplitude value of an ultrasonic wave attributed to vertical vibration propagated through a tibia of a male S who is 22 years old.

### DETAILED DESCRIPTION OF THE PREFERRED EMBODIMENTS

### Gist of Present Invention

In order to realize a quantitative and adequate measurement of a progress stage of osteoporosis without placing a significant burden on a body of a subject, the present inventor considers the following gist of the present invention.

First, the present inventor performed a measurement using ultrasonic waves, not a measurement using X-rays, in order to avoid a placement of significant burden on a body of a subject to whom the measurement is performed.

Subsequently, the present inventor examined the aforementioned experimental result of non-patent document 1 regarding the progress stage of osteoporosis, namely, the stage of osteoporosis-like changes.

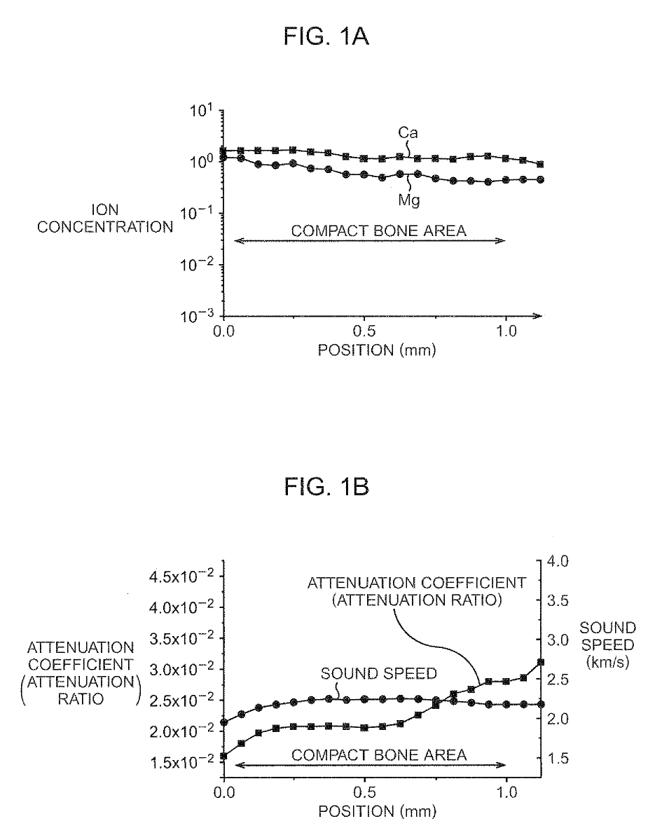

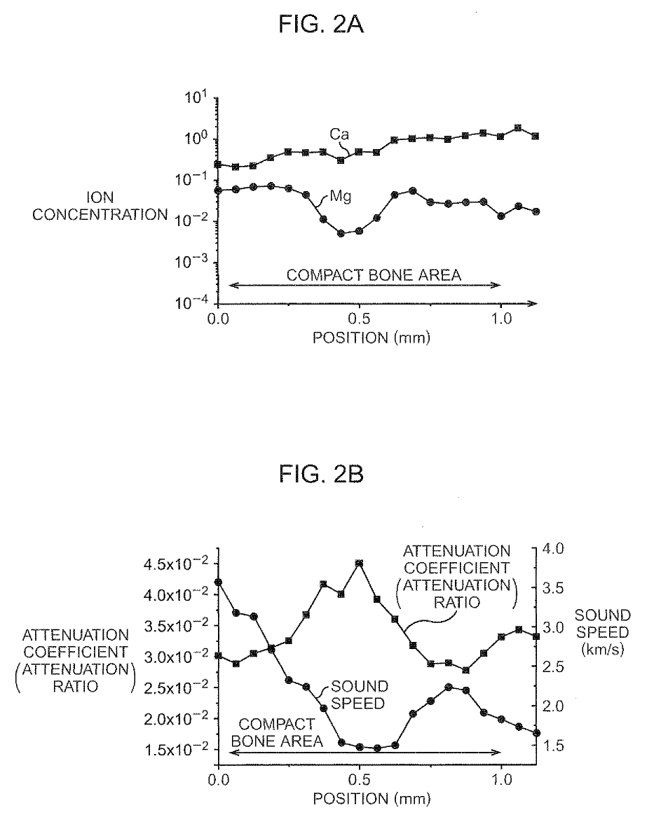

FIG. 1A and FIG. 1B are characteristic diagrams when an ultrasonic wave is applied to the femur of hind limb of the rat in the control group described in Background Art, and FIG. 2A and FIG. 2B are characteristic diagrams when an ultrasonic wave is applied to the femur of hind limb of the rat in the hind-limbs suspended group described in Background Art. Generally, a bone is composed of a periosteum, a compact substance, a spongy substance and a bone marrow cavity in this order from a surface of the bone to the inside thereof, and FIG. 1A and FIG. 1B as well as FIG. 2A and FIG. 2B illustrate the characteristic in which the compact substance in each femur of hind limb is extracted as much as about 1.0 mm.

FIG. 1A and FIG. 2A illustrate a characteristic regarding Ca ions and Mg ions in the compact substance of each femur of hind limb caused by an ion exciting ultrasonic wave, in which a horizontal axis indicates a position in the compact substance from the periosteum side toward the bone marrow cavity, and a vertical axis indicates a concentration of each ion. There is no concentration change in the Ca ions and Mg ions in the compact substance in the femur of hind limb of the rat in the control group shown in FIG. 1A. On the other hand, it can be confirmed that although there is not much concentration change in the Ca ions in the compact substance in the femur of hind limb of the rat in the hind-limbs suspended group shown in FIG. 2A, the concentration of the Mg ions is decreased to about one-tenth in a center part (in the vicinity of 0.5 mm) of the compact substance. It can be read that, in terms of crystallography, a crystal structure in the center part of the compact substance is destroyed.

The present inventor paid attention to that point, and devised to perform, when evaluating the progress stage of osteoporosis (stage of osteoporosis-like changes), new evaluation in accordance with an irreversible Mg deficiency in the inside (particularly the center part of the compact substance) of the bone, with respect to a conventional evaluation in accordance with a decrease in bone density including a spongy substance and the like.

FIG. 1B and FIG. 2B illustrate a characteristic of an attenuation coefficient (attenuation ratio) of an ultrasonic wave propagated through a compact substance of each femur of hind limb and a sound speed of the ultrasonic wave, in which a horizontal axis indicates a position in the compact substance from a periosteum side toward a bone marrow cavity, a left vertical axis indicates the attenuation coefficient (attenuation ratio) of the ultrasonic wave, and a right vertical axis indicates the sound speed of the ultrasonic wave. There is not much rapid change in both the attenuation coefficient and the sound speed in the compact substance in the femur of hind limb of the rat in the control group shown in FIG. 1B. On the other hand, in the compact substance in the femur of hind limb of the rat in the hind-limbs suspended group shown in FIG. 2B, a rapid change was observed in both the attenuation coefficient and the sound speed in the center part (in the vicinity of 0.5 mm) of the compact substance in which a significant decrease in the Mg ion concentration was observed in FIG. 2A.

Further, based on the experimental results shown in FIG. 1B and FIG. 2B, the present inventor found out that when the progress stage of osteoporosis (stage of osteoporosis-like changes) is evaluated, it is effective to conduct evaluation by using an attenuation coefficient (attenuation ratio) of an ultrasonic wave propagated through an inside of a bone part of a subject or a sound speed of the ultrasonic wave as a parameter.

In addition, the present inventor also examined the ultrasonic wave to be applied to the bone part of the subject. Concretely, the present inventor investigated a relationship among a Mg deficiency in the bone part such as a long bone of the subject, an ultrasonic wave that gives vibration in a torsional direction of the bone part (hereinafter, the vibration is referred to as “torsional vibration”), and an ultrasonic wave that gives vibration in a vertical direction (longitudinal direction) of the bone part (hereinafter, the vibration is referred to as “vertical vibration”). As a result of the investigation, it was found that the vertical vibration is hard to be affected by the Mg deficiency compared to the torsional vibration.

Specifically, the present inventor found out that when the progress stage of osteoporosis (stage of osteoporosis-like changes) in the bone part of the subject is evaluated, it is necessary to apply the ultrasonic wave that gives torsional vibration to the bone part to detect the ultrasonic wave propagated through the bone part and to obtain the attenuation coefficient or the sound speed of the ultrasonic wave. Further, the present inventor considered, in order to improve a measurement accuracy of the progress stage of osteoporosis in the bone part of the subject, to detect not only the ultrasonic wave attributed to the torsional vibration but also the ultrasonic wave attributed to the vertical vibration, and to divide each parameter (attenuation coefficient or sound speed) of the ultrasonic wave attributed to the torsional vibration by each parameter of the ultrasonic wave attributed to the vertical vibration.

### Concrete Embodiment of Present Invention

Next, an embodiment of the present invention based on the aforementioned gist of the present invention will be described with reference to the attached drawings.

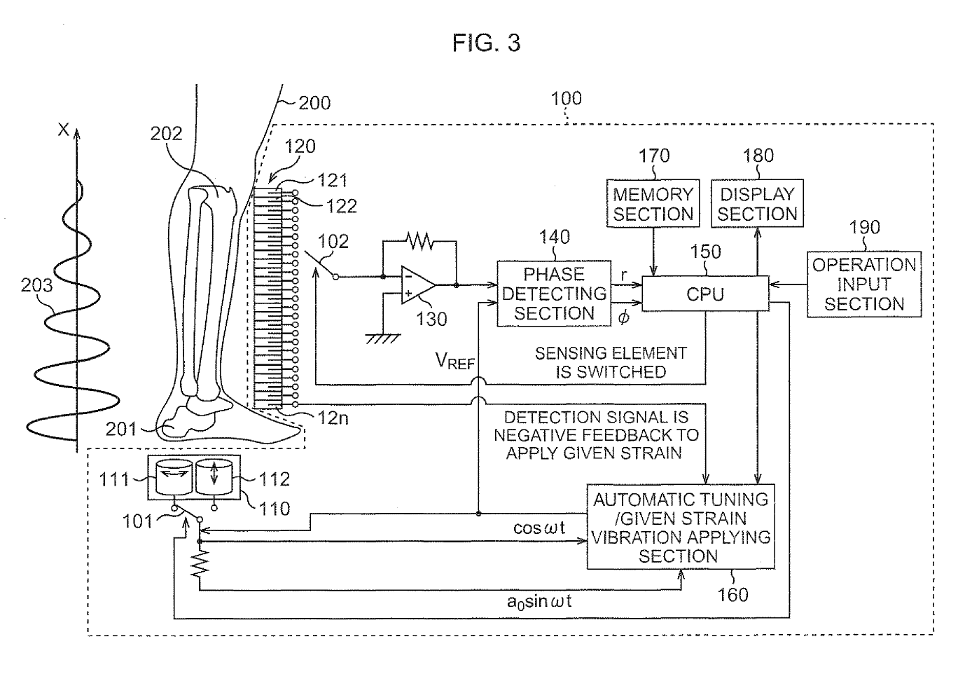

FIG. 3 is a block diagram showing a schematic configuration of an osteoporosis progress stage measuring device 100 according to an embodiment of the present invention.

The osteoporosis progress stage measuring device 100 according to the embodiment of the present invention is configured by including an ultrasonic wave oscillating section 110, an ultrasonic wave detecting section 120, a preamplifier 130, a phase detecting section 140, a CPU 150, an automatic tuning/given strain vibration applying section 160, a memory section 170, a display section 180, an operation input section 190, and switching switches 101 and 102.

The ultrasonic wave oscillating section 110 oscillates an ultrasonic wave that gives vibration to a bone part (calcaneus 201 to tibia 202) of a subject 200. Concretely, the ultrasonic wave oscillating section 110 is provided with a torsional oscillator 111 that oscillates an ultrasonic wave that gives torsional vibration to the calcaneus 201, and a vertical oscillator 112 that oscillates an ultrasonic wave that gives vertical vibration to the calcaneus 201, each being selected when the CPU 150 switches a first switching switch 101.

The ultrasonic wave detecting section 120 detects, through the subject 200, an ultrasonic wave oscillated by the ultrasonic wave oscillating section 110 and propagated through the bone part (calcaneus 201 to tibia 202) of the subject 200. Concretely, the ultrasonic wave detecting section 120 is provided with a plurality of sensing elements (121 to 12n) lined up with respect to the subject 200, in which each sensing element detects an amplitude of ultrasonic wave at each point as an electrical signal. Here, a damped wave 203 in FIG. 3 indicates the ultrasonic wave propagated through the bone part (calcaneus 201 to tibia 202) of the subject 200. Further, a detection signal of each sensing element is selected to be output when the CPU 150 switches a second switching switch 102.

The preamplifier 130 conducts processing such as amplifying the electrical signal of the ultrasonic wave detected by the ultrasonic wave detecting section 120 via the second switching switch 102.

The phase detecting section 140 detects a phase value φ and a phase-correlation amplitude value r of the ultrasonic wave detected by the ultrasonic wave detecting section 120 with respect to the ultrasonic wave oscillated by the ultrasonic wave oscillating section 110.

The CPU 150 comprehensively performs an operation in the osteoporosis progress stage measuring device 100 and a control thereof. For example, the CPU 150 switches the first switching switch 101 based on an input signal from an operator to the operation input section 190 or the like, and switches the second switching switch 102 based on an input signal from the phase detecting section 140 or the like. Further, the CPU 150 calculates a sound speed or an attenuation coefficient (attenuation ratio) of the ultrasonic wave detected by the ultrasonic wave detecting section 120 based on an input signal from the phase detecting section 140, and calculates a characteristic indicating the progress stage of osteoporosis in the bone part of the subject 200 based on the calculated sound speed or the attenuation coefficient (attenuation ratio) of the ultrasonic wave. Further, the CPU 150 also performs control for displaying the calculated characteristic indicating the progress stage of osteoporosis on the display section 180.

In order to vibrate the bone part of the subject 200 at a given strain, the automatic tuning/given strain vibration applying section 160 negative-feedbacks a detection signal from the sensing element 12n located nearest to the ultrasonic wave oscillating section 110 to control a driving voltage that drives the ultrasonic wave oscillating section 110, based on the control by the CPU 150.

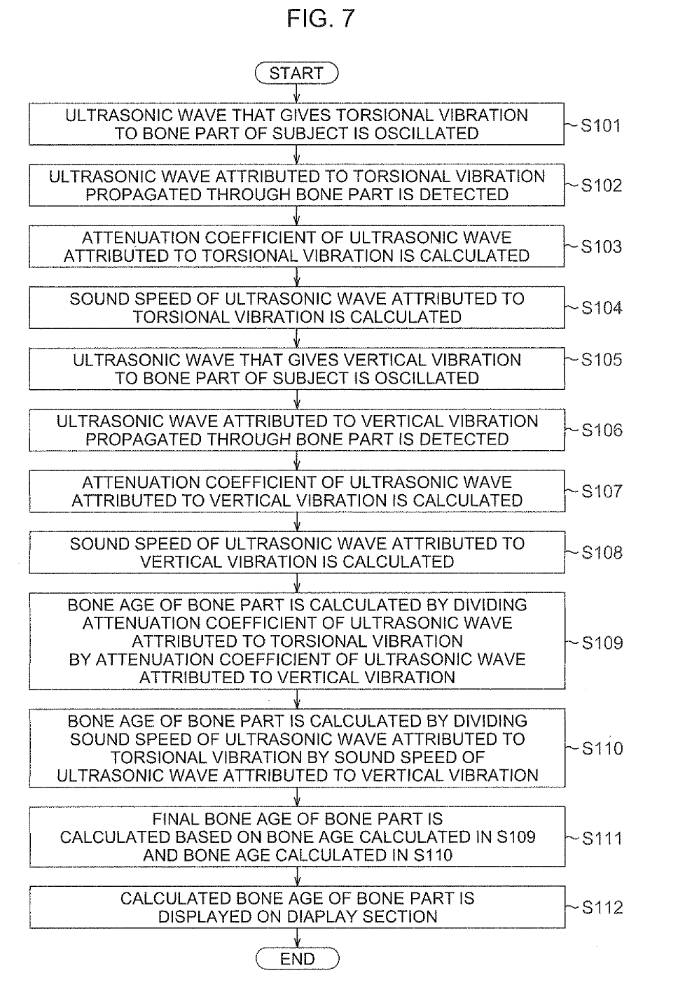

The memory section 170 stores a program necessary for performing processing including later-described processing shown in FIG. 7 conducted by the CPU 150, reference data of osteoporosis progress stage used when the CPU 150 calculates the characteristic indicating the progress stage of osteoporosis in the bone part of the subject 200, and the like. The explanation regarding the reference data of osteoporosis progress stage stored in the memory section 170 will be made hereinbelow.

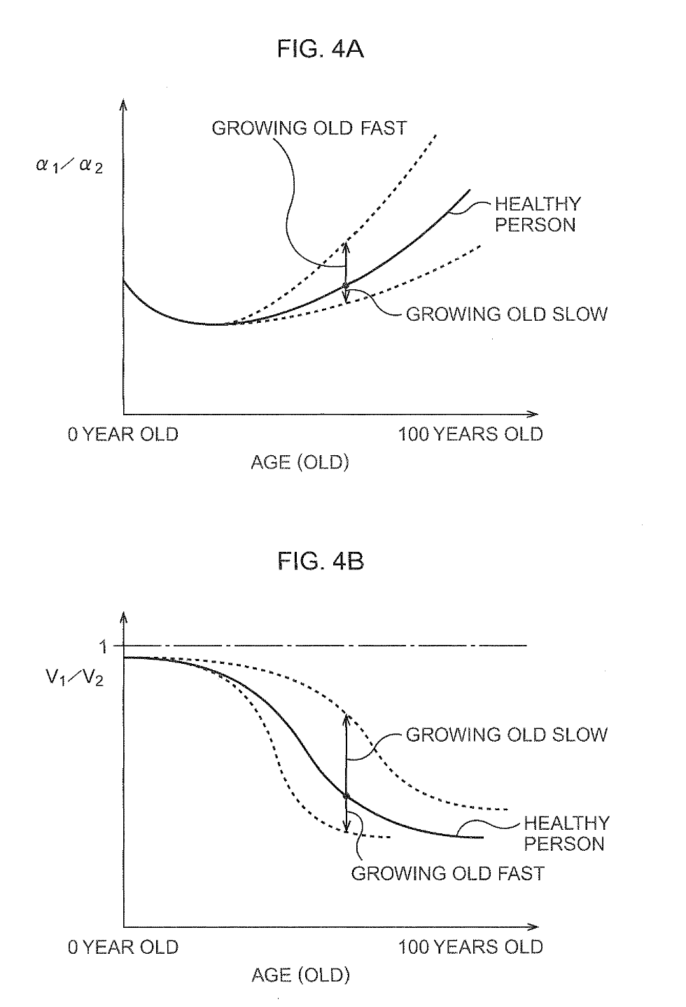

FIG. 4A and FIG. 4B are schematic diagrams showing an example of reference data of osteoporosis progress stage. Here, FIG. 4A and FIG. 4B illustrate a characteristic of bone age as a characteristic indicating the progress stage of osteoporosis. FIG. 4A illustrates a characteristic of healthy person in which a horizontal axis takes the bone age and a vertical axis takes a ratio obtained by dividing an attenuation coefficient α₁ of an ultrasonic wave attributed to torsional vibration by an attenuation coefficient α₂ of an ultrasonic wave attributed to vertical vibration. FIG. 4B illustrates a characteristic of healthy person in which a horizontal axis takes the bone age and a vertical axis takes a ratio obtained by dividing a sound speed V₁ of an ultrasonic wave attributed to torsional vibration by a sound speed V₂ of an ultrasonic wave attributed to vertical vibration.

On the display section 180 in FIG. 3, information indicating a measurement result of bone age of the subject 200 being the characteristic indicating the progress stage of osteoporosis calculated by the CPU 150, an operation status of the osteoporosis progress stage measuring device 100 or the like is displayed by the CPU 150.

The operation input section 190 accepts an operation input from an operator with respect to the osteoporosis progress stage measuring device 100.

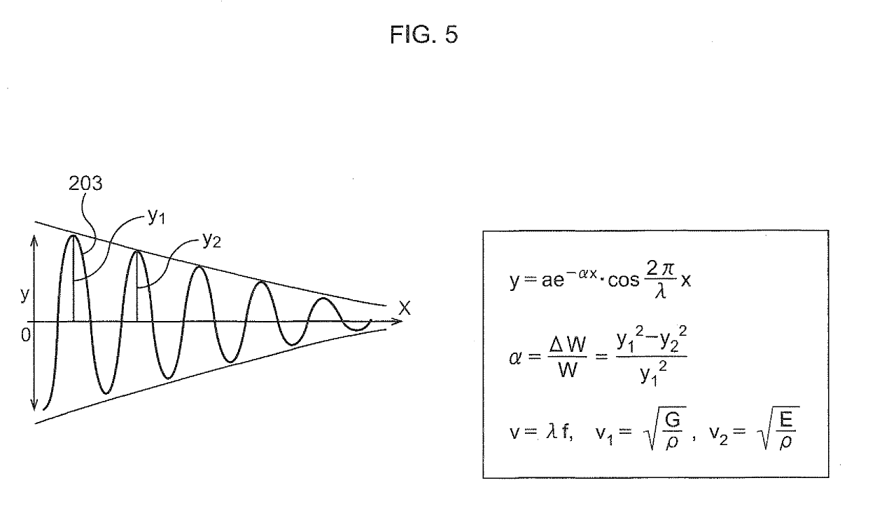

FIG. 5 is a diagram for explaining the damped wave 203 of the ultrasonic wave propagated through the bone part (calcaneus 201 to tibia 202) of the subject 200.

As shown in FIG. 5, when a traveling direction is set to x, an amplitude y of the damped wave 203 can be generally represented by the following expression.

y=aEXP('αx)cos(2π/λ)x. (expression 1)

Here, a, α and λ respectively indicate an external amplitude of the subject 200 closest to the ultrasonic wave oscillating section 110, an attenuation coefficient (attenuation ratio) of the damped wave 203, and a wavelength of the damped wave 203 in a standing wave.

Further, as shown in FIG. 5, the attenuation coefficient α of the damped wave 203 can be represented by the following expression.

α=(y₁²−y₂²)/y₁². (expression 2)

Further, as shown in FIG. 5, a sound speed V of the damped wave 203 can be represented by the following expression.

V=λf. (expression 3)

Here, f is a frequency of the damped wave 203.

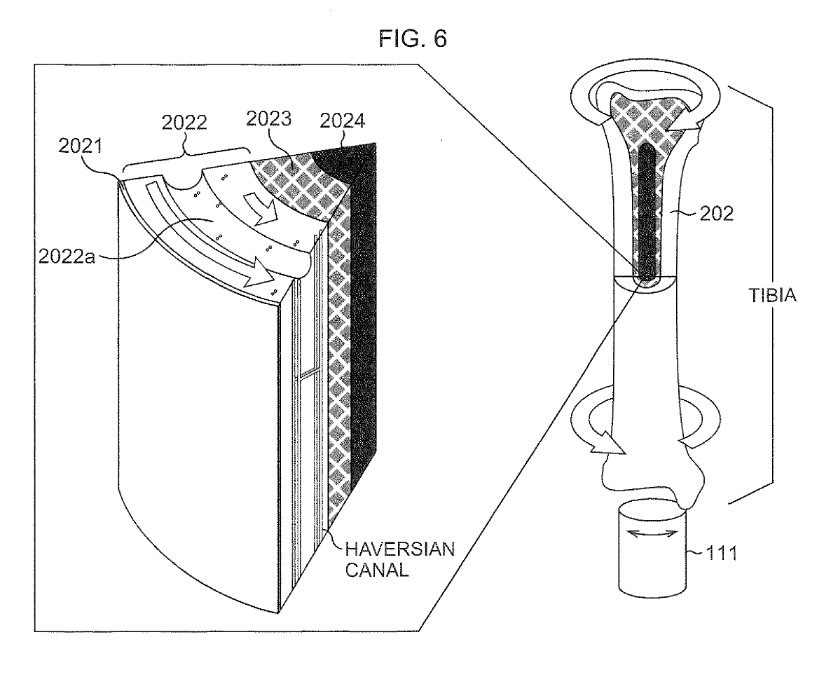

FIG. 6 is a schematic diagram showing a schematic configuration of the tibia 202 shown in FIG. 3. Further, FIG. 6 also illustrates an image when torsional vibration is applied from the torsional oscillator 111 to the tibia 202.

As shown in FIG. 6, the tibia 202 is composed of a periosteum 2021, a compact substance 2022, a spongy substance 2023 and a bone marrow cavity 2024 in this order from a surface of the tibia 202 to the inside thereof, in which a Mg (ion)-deficient part 2022a exists in a center part of the compact substance 2022. Further, a torsional strain caused by the torsional vibration is also illustrated by an arrow in the tibia 202, which indicates that the torsional strain is transmitted through the Mg (ion)-deficient part 2022a.

As described in the aforementioned gist of the present invention, it is confirmed that the vertical vibration is not so much affected by the Mg (ion)-deficient part 2022a as the torsional vibration is. In the present embodiment, in order to absorb a variation due to a thickness of tissue between the tibia 202 and a skin caused when measuring the ultrasonic wave propagated through the tibia 202 from an external of a side of a body of the subject 200, each parameter (attenuation coefficient or sound speed) of the ultrasonic wave attributed to the torsional vibration is divided by each parameter of the ultrasonic wave attributed to the vertical vibration, to thereby eliminate an influence of the variation.

Next, a processing procedure of an osteoporosis progress stage measuring method using the osteoporosis progress stage measuring device 100 will be described.

FIG. 7 is a flow chart showing the processing procedure of the osteoporosis progress stage measuring method using the osteoporosis progress stage measuring device 100 according to the embodiment of the present invention.

First, in step S101, the CPU 150 turns the first switching switch 101 to the torsional oscillator 111 side, to thereby make the torsional oscillator 111 oscillate an ultrasonic wave that gives torsional vibration to the bone part (calcaneus 201 to tibia 202) of the subject 200. At this time, the automatic tuning/given strain vibration applying section 160 attunes the ultrasonic wave oscillated from the torsional oscillator 111 to a resonant frequency so that the ultrasonic wave attributed to the torsional vibration propagated through the bone part (calcaneus 201 to tibia 202) of the subject 200 becomes a resonant state, and further, it negative-feedbacks the detection signal from the sensing element 12n to supply the driving voltage for vibrating the bone part of the subject 200 at a given strain to the torsional oscillator 111.

Next, in step S102, the ultrasonic wave detecting section 120 detects, through the subject 200, the ultrasonic wave oscillated from the torsional oscillator 111 and attributed to the torsional vibration propagated through the bone part (calcaneus 201 to tibia 202) of the subject 200.

Subsequently, in step S103, the CPU 150 calculates the attenuation coefficient (attenuation ratio) al of the ultrasonic wave attributed to the torsional vibration detected by the ultrasonic wave detecting section 120 with respect to the ultrasonic wave oscillated from the torsional oscillator 111.

Concretely, in step S103, the phase detecting section 140 first detects a phase value φ and a phase-correlation amplitude value r of the ultrasonic wave attributed to the torsional vibration detected by each of the sensing elements (121 to 12n) of the ultrasonic wave detecting section 120.

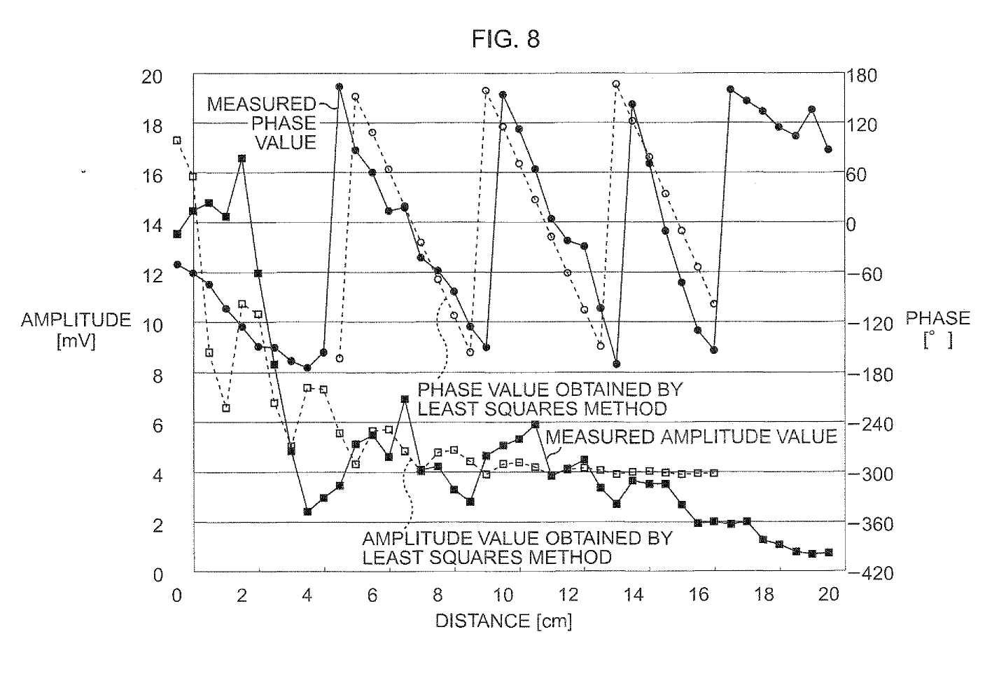

FIG. 8 is a characteristic diagram showing a phase value φ and a phase-correlation amplitude value r of an ultrasonic wave attributed to torsional vibration propagated through a tibia of a male S who is 22 years old. In FIG. 8, a horizontal axis indicates a distance between each sensing element and the sensing element 12n, a left vertical axis indicates the phase-correlation amplitude value r, and a right vertical axis indicates the phase value φ. FIG. 8 shows a measurement value of each of the phase values φ and phase-correlation amplitude values r, and a calculation value of each of the phase values φ and phase-correlation amplitude values r calculated through fitting with the use of a least squares method.

The ultrasonic wave attributed to the torsional vibration sampled at intervals by each of the sensing elements of the ultrasonic wave detecting section 120 is approximated, in the CPU 150, to the aforementioned expression 1 by the least squares method, and the attenuation coefficient (attenuation ratio) α₁ of the ultrasonic wave attributed to the torsional vibration and a wavelength λᵢ of the ultrasonic wave are calculated. For instance, in an example of FIG. 8, the attenuation coefficient (attenuation ratio) α₁ is calculated to be 0.32 and the wavelength λ₁ is calculated to be 4.1 cm.

Next, in step S104, the CPU 150 calculates, based on the ultrasonic wave attributed to the torsional vibration detected by the ultrasonic wave detecting section 120, the sound speed V₁ of the ultrasonic wave.

Concretely, in step S104, the sound speed V₁ of the ultrasonic wave attributed to the torsional vibration is calculated using the expression 3. For example, if a frequency f₁ of the ultrasonic wave attributed to the torsional vibration propagated through the bone part of the subject 200 is 53.66 kHz, by using a value of the wavelength λ₁ calculated together with the attenuation coefficient α₁ in step S103, the value of the wavelength λ₁ and the value of the frequency f₁ are substituted into the expression 3, resulting that the sound speed V₁ is calculated to be 2200 m/s.

Subsequently, in step S105, the CPU 150 turns the first switching switch 101 to the vertical oscillator 112 side, to thereby make the vertical oscillator 112 oscillate an ultrasonic wave that gives vertical vibration to the bone part (calcaneus 201 to tibia 202) of the subject 200. At this time, the automatic tuning/given strain vibration applying section 160 attunes the ultrasonic wave oscillated from the vertical oscillator 112 to a resonant frequency so that the ultrasonic wave attributed to the vertical vibration propagated through the bone part (calcaneus 201 to tibia 202) of the subject 200 becomes a resonant state, and further, it negative-feedbacks the detection signal from the sensing element 12n to supply the driving voltage for vibrating the bone part of the subject 200 at a given strain to the vertical oscillator 112.

Next, in step S106, the ultrasonic wave detecting section 120 detects, through the subject 200, the ultrasonic wave oscillated from the vertical oscillator 112 and attributed to the vertical vibration propagated through the bone part (calcaneus 201 to tibia 202) of the subject 200.

Subsequently, in step S107, the CPU 150 calculates the attenuation coefficient (attenuation ratio) α₂ of the ultrasonic wave attributed to the vertical vibration detected by the ultrasonic wave detecting section 120 with respect to the ultrasonic wave oscillated from the vertical oscillator 112.

Concretely, in step S107, the phase detecting section 140 first detects a phase value φ and a phase-correlation amplitude value r of the ultrasonic wave attributed to the vertical vibration detected by each of the sensing elements (121 to 12n) of the ultrasonic wave detecting section 120.

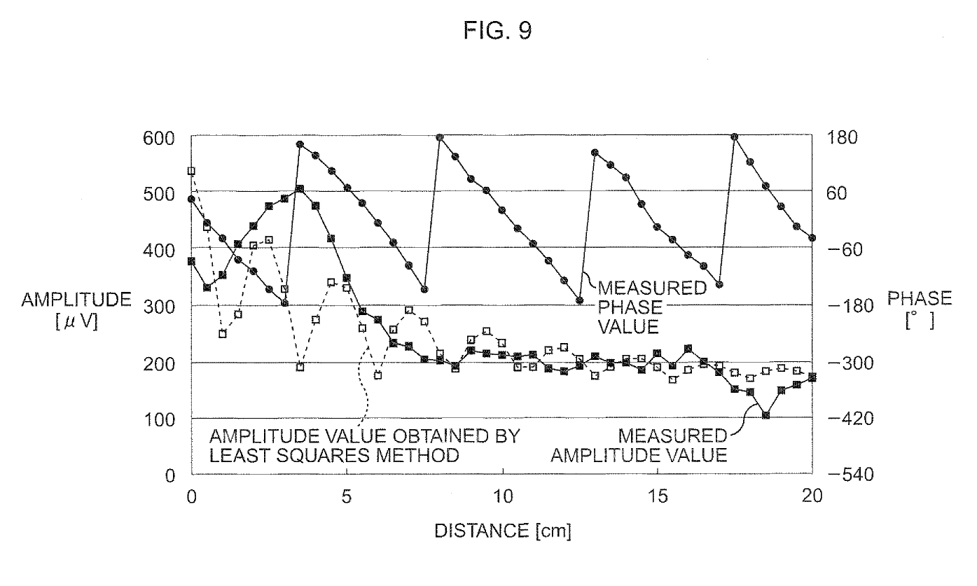

FIG. 9 is a characteristic diagram showing a phase value φ and a phase-correlation amplitude value r of an ultrasonic wave attributed to vertical vibration propagated through a tibia of a male S who is 22 years old. In FIG. 9, a horizontal axis indicates a distance between each sensing element and the sensing element 12n, a left vertical axis indicates the phase-correlation amplitude value r, and a right vertical axis indicates the phase value φ. FIG. 9 shows a measurement value of each of the phase values φ and phase-correlation amplitude values r, and a calculation value of each of the phase-correlation amplitude values r calculated through fitting with the use of a least squares method.

The ultrasonic wave attributed to the vertical vibration sampled at intervals by each of the sensing elements of the ultrasonic wave detecting section 120 is approximated, in the CPU 150, to the aforementioned expression 1 by the least squares method, and the attenuation coefficient (attenuation ratio) α₂ of the ultrasonic wave attributed to the vertical vibration and a wavelength λ₂ of the ultrasonic wave are calculated. For instance, in an example of FIG. 9, the attenuation coefficient (attenuation ratio) α₂ is calculated to be 0.16 and the wavelength λ₂ is calculated to be 4.8 cm. Here, in examples of FIG. 8 and FIG. 9, the attenuation coefficient α₁ of the ultrasonic wave attributed to the torsional vibration is calculated to be 0.32 and the attenuation coefficient α₂ of the ultrasonic wave attributed to the vertical vibration is calculated to be 0.16. As confirmed from the above, the torsional vibration is more likely to be affected by the Mg (ion)-deficient part 2022a.

Next, in step S108, the CPU 150 calculates, based on the ultrasonic wave attributed to the vertical vibration detected by the ultrasonic wave detecting section 120, the sound speed V₂ of the ultrasonic wave.

Concretely, in step S108, the sound speed V₂ of the ultrasonic wave attributed to the vertical vibration is calculated using the expression 3. For example, if a frequency f₂ of the ultrasonic wave attributed to the vertical vibration propagated through the bone part of the subject 200 is 39.58 kHz, by using a value of the wavelength λ2 calculated together with the attenuation coefficient α₂ in step S107, the value of the wavelength λ₂ and the value of the frequency f₂ are substituted into the expression 3, resulting that the sound speed V₂ is calculated to be 1900 m/s.

Subsequently, in step S109, the CPU 150 calculates, based on the calculated attenuation coefficient of the ultrasonic wave, a bone age of the bone part of the subject 200 as a characteristic indicating the progress stage of osteoporosis.

Concretely, in step S109, a ratio obtained by dividing the attenuation coefficient α₁ of the ultrasonic wave attributed to the torsional vibration calculated in step S103 by the attenuation coefficient α₂ of the ultrasonic wave attributed to the vertical vibration calculated in step S107 (α₁/α₂) is first determined. Subsequently, by referring to the reference data of osteoporosis progress stage shown in FIG. 4A stored in the memory section 170, a bone age of healthy person corresponding to the determined ratio (α₁/α₂) is calculated. At this time, the calculated bone age is supposed to be 32 years.

In the reference data of osteoporosis progress stage shown in FIG. 4A, a value of ratio (α₁/α₂) tends to be increased with an increase in age. Accordingly, for instance, when a value of the actually measured ratio (α₁/α₂) is larger than a value of ratio (α₁/α₂) corresponding to an actual age of the subject 200, the bone age of the subject 200 is older than the actual age (this is referred to as “growing old fast”), and meanwhile, when the value of the actually measured ratio (α₁/α₂) is smaller than the value of ratio (α₁/α₂) corresponding to the actual age of the subject 200, the bone age of the subject 200 is younger than the actual age (this is referred to as “growing old slow”).

Subsequently, in step S110, the CPU 150 calculates, based on the calculated sound speed of the ultrasonic wave, a bone age of the bone part of the subject 200 as a characteristic indicating the progress stage of osteoporosis.

Concretely, in step S110, a ratio obtained by dividing the sound speed V₁ of the ultrasonic wave attributed to the torsional vibration calculated in step S104 by the sound speed V₂ of the ultrasonic wave attributed to the vertical vibration calculated in step S108 (V₁/V₂) is first determined. Subsequently, by referring to the reference data of osteoporosis progress stage shown in FIG. 4B stored in the memory section 170, a bone age of healthy person corresponding to the determined ratio (V₁/V₂) is calculated. At this time, the calculated bone age is supposed to be 34 years.

In the reference data of osteoporosis progress stage shown in FIG. 4B, a value of ratio (V₁/V₂) tends to be decreased with an increase in age. Accordingly, for instance, when a value of the actually measured ratio (V₁/V₂) is smaller than a value of ratio (V₁/V₂) corresponding to an actual age of the subject 200, the bone age of the subject 200 grows old fast with respect to the actual age, and meanwhile, when the value of the actually measured ratio (V₁/V₂) is larger than the value of ratio (V₁/V₂) corresponding to the actual age of the subject 200, the bone age of the subject 200 grows old slow with respect to the actual age.

Next, in step S111, the CPU 150 calculates a final bone age of the bone part of the subject 200 based on the bone age calculated in step S109 and the bone age calculated in step S110.

Concretely, since the bone age is calculated to be 32 years in step S109 and it is calculated to be 34 years in step S110, averaging processing is performed in step S111, resulting that the final bone age of the bone part of the subject 200 is calculated to be 33 years.

Subsequently, in step S112, the CPU 150 displays the bone age of the bone part of the subject 200 calculated in step S111 on the display section 180. Accordingly, it becomes possible to quantitatively grasp the progress stage of osteoporosis in the bone part of the subject 200 based on the displayed bone age.

Through the above processing of step S101 to step S112, the bone age of the bone part of the subject 200 is calculated based on the attenuation coefficient and the sound speed of the ultrasonic wave propagated through the bone part of the subject 200, and the display of the bone age is performed.

Note that in the aforementioned embodiment, both the attenuation coefficient and the sound speed of the ultrasonic wave propagated through the bone part of the subject 200 are designed to be calculated for the purpose of further improving the measurement accuracy of the characteristic indicating the progress stage of osteoporosis (bone age). However, an embodiment in which the characteristic indicating the progress stage of osteoporosis (bone age) is calculated by using either the attenuation coefficient or the sound speed to simplify the processing, is also included in the present invention.

Concretely, for instance, when the calculation of bone age of the bone part is performed by using only the attenuation coefficient of the ultrasonic wave propagated through the bone part of the subject 200, an embodiment is taken in which the respective steps of steps S101 to S103, S105 to S107, S109 and S112 in FIG. 7 are conducted. Further, for instance, when the calculation of bone age of the bone part is performed by using only the sound speed of the ultrasonic wave propagated through the bone part of the subject 200, an embodiment is taken in which the respective steps of steps S101, S102, S104 to S106, 5108, 5110, and 5112 in FIG. 7 are conducted.

Further, in the aforementioned embodiment, the detection of the ultrasonic wave attributed to the vertical vibration is designed to be conducted in addition to the detection of the ultrasonic wave attributed to the torsional vibration propagated through the bone part of the subject 200 for the purpose of further improving the measurement accuracy of the characteristic indicating the progress stage of osteoporosis (bone age). However, an embodiment in which only the detection of the ultrasonic wave attributed to the torsional vibration is performed to simplify the processing, is also included in the present invention.

Concretely, when the calculation of bone age of the bone part of the subject 200 is performed by conducting only the detection of the ultrasonic wave attributed to the torsional vibration, each piece of the reference data of osteoporosis progress stage shown in FIG. 4A and FIG. 4B as the reference data of osteoporosis progress stage previously prepared in the memory section 170 is set to illustrate a characteristic of bone age in which the vertical axes thereof are changed to indicate the attenuation coefficient α₁ of the ultrasonic wave attributed to the torsional vibration and the sound speed V₁ of the ultrasonic wave attributed to the torsional vibration, respectively. Further, in this case, after conducting the processing of steps S101 to S104 in FIG. 7, the bone age of the bone part of the subject 200 is calculated, in step S109, from the reference data of osteoporosis progress stage stored in the memory section 170 based on the attenuation coefficient α₁ of the ultrasonic wave attributed to the torsional vibration calculated in step S103. Subsequently, in step S110, the bone age of the bone part of the subject 200 is calculated from the reference data of osteoporosis progress stage stored in the memory section 170 based on the sound speed V₁ of the ultrasonic wave attributed to the torsional vibration calculated in step S104, and further, the respective steps of step S111 and step S112 are conducted, and such an embodiment described above is taken.

Further, in the aforementioned embodiment, the ratio (V₁/V₂) obtained by dividing the sound speed V₁ of the ultrasonic wave attributed to the torsional vibration calculated in step S104 by the sound speed V₂ of the ultrasonic wave attributed to the vertical vibration calculated in step S108 is designed to be determined to calculate the bone age of healthy person corresponding to the determined ratio (V₁/V₂) by refereeing to the reference data of osteoporosis progress stage shown in FIG. 4B in step S110. However, for instance, an embodiment in which a Poisson's ratio in the bone part of the subject 200 is determined to calculate the bone age of healthy person, can also be applied to the present invention. The explanation regarding the Poisson's ratio will be given hereinbelow.

Here, as shown in FIG. 5, the sound speed V₁ of the ultrasonic wave attributed to the torsional vibration propagated through a medium with a density ρ and the sound speed V₂ of the ultrasonic wave attributed to the vertical vibration propagated through the medium with the density ρ can be respectively represented by the following expression 4 and expression 5, in which a modulus in torsion (modulus of shear elasticity) of the medium and a Young's modulus of the medium are set to G and E, respectively.

V₁=·(G/ρ). (expression 4)

V₂=·(E/ρ). (expression 5)

Further, a Poisson's ratio ν can be generally represented by the following expression 6.

G=E/{2(1+v)}. (expression 6)

Accordingly, from the expression 4 to expression 6, the Poisson's ratio ν can be represented by the following expression 7.

$$\begin{matrix}v = E / ( 2 G ) - 1 \\ = ( 1 / 2 ) \cdot {( {V}_{2} / {V}_{1} )}^{2} - 1.\end{matrix} \qquad ( expression 7 )$$

In the embodiment using the Poisson's ratio ν, reference data of osteoporosis progress stage regarding a characteristic of healthy person in which a horizontal axis takes a bone age and a vertical axis takes the Poisson's ratio ν of the bone part of the subject 200, is previously prepared in the memory section 170. Here, the reference data of osteoporosis progress stage using the Poisson's ratio ν corresponds to the reference data of osteoporosis progress stage using the ratio (V₁/V₂) between the sound speed V₁ and the sound speed V₂ shown in FIG. 4B which is modified based on the correlation shown by the expression 7.

Concretely, in the embodiment using the Poisson's ratio v, the Poisson's ratio ν is first determined using the expression 7 based on the sound speed V₁ of the ultrasonic wave attributed to the torsional vibration calculated in step S104 and the sound speed V₂ of the ultrasonic wave attributed to the vertical vibration calculated in step S108. Subsequently, by referring to the reference data of osteoporosis progress stage using the Poisson's ratio ν stored in the memory section 170, a bone age of healthy person corresponding to the determined Poisson's ratio ν is calculated. By the above, the embodiment in which the bone age of healthy person is calculated using the Poisson's ratio is conducted.

Further, in the aforementioned embodiment, the attenuation coefficient and the sound speed of the ultrasonic wave are calculated by using each of the detection signals of each of the sensing elements (121 to 12n) of the ultrasonic wave detecting section 120 by switching the second switching switch 102. However, for instance, it is also possible to continuously obtain a detection signal of a certain sensing element without switching the second switching switch 102, and to calculate the attenuation coefficient and the sound speed based on an ultrasonic waveform at a position of the sensing element, to thereby simplify the processing. In this case, the attenuation coefficient of the ultrasonic wave is calculated based on the ultrasonic waveform at the position of the sensing element with respect to a waveform of ultrasonic wave oscillated by the ultrasonic wave oscillating section 110. Further, the sound speed of the ultrasonic wave is calculated based on the ultrasonic waveform detected by the sensing element.

Besides, in the aforementioned embodiment, an explanation is made by citing a case of a human being (human body) as an example of the subject 200, but, the present invention is not limited to this and, for example, another animal such as a rat, a cat and a dog can also be applied as the subject 200.

According to the present embodiment, the bone age being the characteristic indicating the progress stage of osteoporosis is designed to be calculated by detecting the ultrasonic wave propagated through the bone part of the subject 200 and using at least either of the attenuation coefficient and the sound speed of the ultrasonic wave, so that it becomes possible to quantitatively and adequately measure the progress stage of osteoporosis without placing a significant burden on the body of the subject.

The respective means in FIG. 3 composing the osteoporosis progress stage measuring device according to the aforementioned present embodiment and the respective steps in FIG. 7 indicating the osteoporosis progress stage measuring method can be realized by operating a program stored in the memory section 170.

The program and a computer readable storage medium (memory section 170) recording the program are within the scope of the present invention.

Specifically, the program is recorded in, for example, the storage medium such as a CD-ROM, or provided to the computer via various transmission media. As a storage medium recording the program, a flexible disk, a hard disk, a magnetic tape, a magneto-optical disk, a nonvolatile memory card, and the like can be used in addition to the CD-ROM. Meanwhile, as a transmission medium of the program, a communication medium in a computer network (LAN, WAN such as the Internet, a wireless communication network, and the like) system to supply the program information by propagating it as a carrier wave can be utilized. Further, as a communication medium at this time, a wired circuit such as an optical fiber, a wireless circuit, or the like can be cited.

Further, the present invention is not limited to an example in which the computer executes the supplied program to realize a function of the osteoporosis progress stage measuring device according to the present embodiment. Also when the program collaborates with an OS (operating system), another application software, or the like, which are operating in the computer, to realize the function of the osteoporosis progress stage measuring device according to the present embodiment, such a program is within a scope of the present invention. Further, when all or parts of the processing of the supplied program are performed by a function expansion board or a function expansion unit of the computer to realize the function of the osteoporosis progress stage measuring device according to the present embodiment, such a program is within the scope of the present invention.

Further, the present embodiments are to be considered in all respects as illustrative and no restrictive, and all changes which come within the meaning and range of equivalency of the claims are therefore intended to be embraced therein. The invention may be embodied in other specific forms without departing from the spirit or essential characteristics thereof.

### INDUSTRIAL APPLICABILITY

According to the present invention, it is possible to quantitatively and adequately measure a progress stage of osteoporosis without placing a significant burden on a body of a subject.

1-13. (canceled)

14. An osteoporosis progress stage measuring device, comprising:
an oscillating unit oscillating an ultrasonic wave that gives torsional vibration to a bone part of a subject and an ultrasonic wave that gives vertical vibration to the bone part;
a detecting unit detecting, through the subject, an ultrasonic wave attributed to the torsional vibration propagated through the bone part and an ultrasonic wave attributed to the vertical vibration propagated through the bone part;
a sound speed calculating unit calculating a sound speed of the ultrasonic wave attributed to the torsional vibration based on the ultrasonic wave attributed to the torsional vibration detected by said detecting unit, and a sound speed of the ultrasonic wave attributed to the vertical vibration based on the ultrasonic wave attributed to the vertical vibration detected by said detecting unit; and
an osteoporosis progress stage characteristic calculating unit calculating a characteristic indicating the progress stage of osteoporosis in the bone part by dividing the sound speed of the ultrasonic wave attributed to the torsional vibration by the sound speed of the ultrasonic wave attributed to the vertical vibration calculated by said sound speed calculating unit.

15. An osteoporosis progress stage measuring device, comprising:
an oscillating unit oscillating an ultrasonic wave that gives torsional vibration to a bone part of a subject and an ultrasonic wave that gives vertical vibration to the bone part;
a detecting unit detecting, through the subject, an ultrasonic wave attributed to the torsional vibration propagated through the bone part and an ultrasonic wave attributed to the vertical vibration propagated through the bone part;
a sound speed calculating unit calculating a sound speed of the ultrasonic wave attributed to the torsional vibration based on the ultrasonic wave attributed to the torsional vibration detected by said detecting unit, and a sound speed of the ultrasonic wave attributed to the vertical vibration based on the ultrasonic wave attributed to the vertical vibration detected by said detecting unit; and
an osteoporosis progress stage characteristic calculating unit calculating a characteristic indicating the progress stage of osteoporosis in the bone part by determining a Poisson's ratio using the sound speed of the ultrasonic wave attributed to the torsional vibration and the sound speed of the ultrasonic wave attributed to the vertical vibration calculated by said sound speed calculating unit.

16. The osteoporosis progress stage measuring device according to claim 14, further comprising
an attenuation coefficient calculating unit calculating an attenuation coefficient of the ultrasonic wave detected by said detecting unit with respect to the ultrasonic wave oscillated by said oscillating unit,
wherein said osteoporosis progress stage characteristic calculating unit calculates the characteristic indicating the progress stage of osteoporosis in the bone part based on the sound speed calculated by said sound speed calculating unit and the attenuation coefficient calculated by said attenuation coefficient calculating unit.

17. An osteoporosis progress stage measuring device, comprising:
an oscillating unit oscillating an ultrasonic wave that gives torsional vibration to a bone part of a subject;
a detecting unit detecting, through the subject, an ultrasonic wave attributed to the torsional vibration propagated through the bone part;
an attenuation coefficient calculating unit calculating an attenuation coefficient of the ultrasonic wave attributed to the torsional vibration detected by said detecting unit with respect to the ultrasonic wave oscillated by said oscillating unit; and
an osteoporosis progress stage characteristic calculating unit calculating, based on the attenuation coefficient calculated by said attenuation coefficient calculating unit, a characteristic indicating the progress stage of osteoporosis in the bone part.

18. The osteoporosis progress stage measuring device according to claim 17,
wherein said oscillating unit further oscillates an ultrasonic wave that gives vertical vibration to the bone part;
wherein said detecting unit further detects, through the subject, an ultrasonic wave attributed to the vertical vibration propagated through the bone part;
wherein said attenuation coefficient calculating unit further calculates an attenuation coefficient of the ultrasonic wave attributed to the vertical vibration detected by said detecting unit with respect to the ultrasonic wave oscillated by said oscillating unit; and
wherein said osteoporosis progress stage characteristic calculating unit calculates the characteristic indicating the progress stage of osteoporosis in the bone part by dividing the attenuation coefficient of the ultrasonic wave attributed to the torsional vibration by the attenuation coefficient of the ultrasonic wave attributed to the vertical vibration calculated by said attenuation coefficient calculating unit.

19. The osteoporosis progress stage measuring device according to claim 17, further comprising
a sound speed calculating unit calculating, based on the ultrasonic wave detected by said detecting unit, a sound speed of the ultrasonic wave,
wherein said osteoporosis progress stage characteristic calculating unit calculates the characteristic indicating the progress stage of osteoporosis in the bone part based on the sound speed calculated by said sound speed calculating unit and the attenuation coefficient calculated by said attenuation coefficient calculating unit.

20. The osteoporosis progress stage measuring device according to claim 14, further comprising
a display unit displaying the characteristic calculated by said osteoporosis progress stage characteristic calculating unit.

21. The osteoporosis progress stage measuring device according to claim 17, further comprising
a display unit displaying the characteristic calculated by said osteoporosis progress stage characteristic calculating unit.

22. The osteoporosis progress stage measuring device according to claim 14,
wherein the characteristic calculated by said osteoporosis progress stage characteristic calculating unit is a bone age of the bone part.

23. The osteoporosis progress stage measuring device according to claim 17,
wherein the characteristic calculated by said osteoporosis progress stage characteristic calculating unit is a bone age of the bone part.

24. An osteoporosis progress stage measuring method, comprising:
an oscillating step of oscillating an ultrasonic wave that gives torsional vibration to a bone part of a subject and an ultrasonic wave that gives vertical vibration to the bone part;
a detecting step of detecting, through the subject, an ultrasonic wave attributed to the torsional vibration propagated through the bone part and an ultrasonic wave attributed to the vertical vibration propagated through the bone part;
a sound speed calculating step of calculating a sound speed of the ultrasonic wave attributed to the torsional vibration based on the ultrasonic wave attributed to the torsional vibration detected by said detecting step, and a sound speed of the ultrasonic wave attributed to the vertical vibration based on the ultrasonic wave attributed to the vertical vibration detected by said detecting step; and
an osteoporosis progress stage characteristic calculating step of calculating a characteristic indicating the progress stage of osteoporosis in the bone part by dividing the sound speed of the ultrasonic wave attributed to the torsional vibration by the sound speed of the ultrasonic wave attributed to the vertical vibration calculated by said sound speed calculating step.

25. An osteoporosis progress stage measuring method, comprising:
an oscillating step of oscillating an ultrasonic wave that gives torsional vibration to a bone part of a subject;
a detecting step of detecting, through the subject, an ultrasonic wave attributed to the torsional vibration propagated through the bone part;
an attenuation coefficient calculating step of calculating an attenuation coefficient of the ultrasonic wave attributed to the torsional vibration detected by said detecting step with respect to the ultrasonic wave oscillated by said oscillating step; and
an osteoporosis progress stage characteristic calculating step of calculating, based on the attenuation coefficient calculated by said attenuation coefficient calculating step, a characteristic indicating the progress stage of osteoporosis in the bone part.
